(19) United States
(12) Patent Application Publication
(10) Pub. No.: US 2015/0173884 A1
(43) Pub. Date: Jun. 25, 2015

(54) EXTENDED ANCHOR ENDOLUMINAL FILTER

(71) Applicants: Eric JOHNSON, Woodside, CA (US); Jeremy STIGALL, Carlsbad, CA (US)

(72) Inventors: Eric JOHNSON, Woodside, CA (US); Jeremy STIGALL, Carlsbad, CA (US)

(21) Appl. No.: 14/575,935

(22) Filed: Dec. 18, 2014

Related U.S. Application Data
(60) Provisional application No. 61/918,520, filed on Dec. 19, 2013.

(51) Int. Cl. A61F 2/01

(52) U.S. Cl. CPC A61F 2/01; A61F 2230/0067

(57) ABSTRACT

Endoluminal filters are disclose with a first support member having a first end and a second end and a second support member attached to the first end of the first support member or the second end of the first support member. The filters also include a material capture structure extending between the first and second support members, a crossover between the first and second support members, and the first end or the second end of the first support member. The filters also include at least one tissue anchor with an elongate portion and a curved portion configured to engage with a lumen wall. The elongate portion has a length greater than a length of the curved portion. Methods for deploying and retrieving endoluminal filters are also disclosed.

### CROSS REFERENCE TO RELATED APPLICATIONS?

This application claims priority to U.S. Provisional Patent Application No. 61/918,520, filed on Dec. 19, 2013, and titled “EXTENDED ANCHOR ENDOLUMINAL FILTER,” which is herein incorporated by reference in its entirety.

This application may be related to U.S. patent application Ser. No. 11/969,827 filed on Jan. 4, 2008, titled “ENDOLUMINAL FILTER WITH FIXATION,” and published as U.S. Patent Application Publication No. 2008/0147111 which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This invention relates generally to devices and methods for providing filtration of debris within a body lumen. More particularly, the invention provides a retrievable filter placed percutaneously in the vasculature of a patient to prevent passage of emboli. Additionally, embodiments of the invention provide a filter that can be atraumatically positioned and subsequently removed percutaneously from a blood vessel.

### BACKGROUND

Embolic protection is utilized throughout the vasculature to prevent the potentially fatal passage of embolic material in the bloodstream to smaller vessels where it can obstruct blood flow. The dislodgement of embolic material is often associated with procedures which open blood vessels to restore natural blood flow such as stenting, angioplasty, arthrectomy, endarterectomy or thrombectomy. Used as an adjunct to these procedures, embolic protection devices trap debris and provide a means for removal for the body.

One widely used embolic protection application is the placement of filtration means in the vena cava. Vena cava filters (VCF) prevent the passage of thrombus from the deep veins of the legs into the blood stream and ultimately to the lungs. This condition is known as deep vein thrombosis (DVT), which can cause a potentially fatal condition known as pulmonary embolism (PE).

The first surgical treatment for PE, performed by John Hunter in 1874, was femoral vein ligation. The next major advancement, introduced in the 1950's, was the practice of compartmentalizing of the vena cava using clips, suture or staples. While effective at preventing PE, these methods were associated with significant mortality and morbidity (see, e.g., Kinney TB, Update on inferior vena cava filters, JVIR 2003; 14:425-440, incorporated herein by reference).

A major improvement in PE treatment, in which venous blood flow was maintained, was presented by DeWesse in 1955. This method was called the “harp-string” filter, as represented in FIG. 1A and FIG. 1B, in which strands of silk suture 12 were sewn across the vena cava 11 in a tangential plane below the renal veins 13 to trap thrombus. Reported clinical results demonstrated the effectiveness of this method in preventing PE and maintaining caval patency. (see, e.g., DeWeese M S, A vena cava filter for the prevention of pulmonary embolism, Arch of Surg 1963; 86:852-868, incorporated herein by reference). Operative mortality associated with all of these surgical treatments remained high and therefore limited their applicability.

The current generation of inferior vena cava (IVC) filters began in 1967 with the introduction of the Mobin-Uddin umbrella 21 (FIG. 1C) which is described in further detail in U.S. Pat. No. 3,540,431. The Greenfield filter (FIG. 1D) was introduced in 1973 and is described in further detail in U.S. Pat. No. 3,952,747. These conical-shaped devices were placed endoluminaly in the IVC and utilized hooks or barbs 20, 30 to pierce the IVC wall and fix the position of the device. A variety of conical-shaped, percutaneously placed vena cava filters, based upon this concept are now available. For example, the TULIP with a filter structure 41 (FIG. 1E) further described in U.S. Pat. No. 5,133,733; the RECOVERY with a filter structure 51 (FIG. 1F) further described in U.S. Pat. No. 6,258,026; and the TRAPESE with a filter structure 61 (FIG. 1G) further described in U.S. Pat. No. 6,443,972.

The next advancement in filters added the element of recoverability. Retrievable filters were designed to allow removal from the patient subsequent to initial placement. Retrievable filters are generally effective at preventing PE yet they have a number of shortcomings, such as, for example: failure of the device to deploy into the vessel properly, migration, perforation of the vessel wall, support structure fracture, retrievability actually limited to specific circumstances, and formation of thrombosis on or about the device.

Problems associated with retrievable, conical-shaped devices, such as those illustrated in FIG. 1D, FIG. 1E and FIG. 1F, have been reported in the medical literature. These reported problems include tilting which makes it difficult to recapture the device and compromises filtration capacity. Hooks 30, 40, 50, 60 used to secure these devices have been reported to perforate the vessel wall, cause delivery complications, and fracture. A partially retrievable system is described in detail in pending U.S. Pat. No. 2004/0186512 (FIG. 1H). In this system, the filter portion 71 can be removed from the support structure 70, but the support structure remains in-vivo. All of these described devices share the common limitation that they can be retrieved from only one end.

Additional retrievable endoluminal filters are disclosed in US 2008/0147111 to Eric Johnson et al. (FIG. 1I). FIG. 1I is a perspective view of an endoluminal filter 89 having a first support member 90 having a first end and a second end and a second support member 91 attached to the first end of the first support member 90 or the second end of the first support member 91. The second support member 91 forms a crossover 92 with the first support member 90. The second support member 91 and the first support member 90 are movable relative to each other at the crossover point 92. A material capture structure 93 extends between the first and second support members 90, 91, the crossover 92, and the first end or the second end of the first support member 90. The filter illustrated in FIG. 1I has a retrieval feature 94 on the first end and a retrieval feature 94 on the second end. Tissue anchor 95 is illustrated on the first support member 90 and the second support member 91. The tissue anchor 95 can improve engagement with a luminal wall. The first support member 90 and second support member 91 can be joined together with a crimp 96.

Each of the above referenced articles, patents and patent applications are incorporated herein in its entirety.

In view of the many shortcomings and challenges that remain in the field of endoluminal filtering, there remains a need for improved retrievable, endoluminal filters.

### SUMMARY OF THE DISCLOSURE

The present invention relates to endoluminal filters and methods for deploying and retrieving endoluminal filters.

In some embodiments endoluminal filters are provided. The endoluminal filters include a first support member having a first end and a second end, a second support member attached to the first end of the first support member or the second end of the first support member and forming a crossover with the first support member, a material capture structure extending between the first and second support members, the crossover, and the first end or the second end of the first support member, and at least one tissue anchor including an elongate portion and a curved portion. The curved portion is configured to engage with a lumen wall. The elongate portion has a length of at least 25% of a length between the first end and second end of the first support member. The at least one tissue anchor is formed on the first support member or the second support member.

In any of the embodiments described herein the endoluminal filters can further include a plurality of second tissue anchors having a curved portion and an elongate portion with the elongate portion shorter than the elongate portion of the at least one tissue anchor.

In any of the embodiments described herein the crossover forms a first loop between the first support member and second support member and a second loop between the first support member and second support member. In any of the embodiments described herein the first loop has a first diameter and the second loop has a second diameter with the second diameter smaller than the first diameter The anchors are formed on the support members forming the second loop. In any of the embodiments described herein the length of the elongate portion is greater than about one-quarter of the second diameter. In any of the embodiments described herein the length of the elongate portion is greater than about one-half of the second diameter.

In any of the embodiments described herein the filter is configured to be placed in a lumen having a lumen diameter and the lumen diameter is greater than the first diameter and second diameter. In any of the embodiments described herein the anchors are configured to extend radially from the first and second support member to contact the lumen.

In any of the embodiments described herein the material capture structure has a windsock configuration

In any of the embodiments described herein the material capture structure extends within a plane defined by the first and second support members, the crossover, and the first end or the second end of the first support member.

In any of the embodiments described herein the filters further include a second crossover between the first support member and second support member.

In any of the embodiments described herein the second support member is attached to the first end of the first support member and the second end of the first support member.

In any of the embodiments described herein the first support member and the second support member are formed from a single wire.

In any of the embodiments described herein the first support member or the second support member forms a retrieval feature.

In any of the embodiments described herein the filters further include a retrieval feature on the first end and a retrieval feature on the second end.

In any of the embodiments described herein the first support member and second support member comprise one or more divots configured to engage with the material capture structure.

In any of the embodiments described herein the elongate portion has a length of at least 40% of the length between the first end and second end of the first support member. In any of the embodiments described herein the elongate portion has a length of at least 50% of the length between the first end and second end of the first support member. In any of the embodiments described herein the elongate portion has a length of at least 75% of the length between the first end and second end of the first support member.

In any of the embodiments described herein the filter has a deployed length measured in a straight line between the first end of the first support member and the second end of the first support member with the elongate portion having a length of at least 50% of the deployed length of the filter.

In some embodiments endoluminal filters are provided. The endoluminal filters include a first support member having a first end and a second end, a second support member having a first end and a second end forming a crossover with the first support member, the first end of the first support member attached to the first end of the second support member, the second end of the first support member attached to the second end of the second support member, the first support member and second support member defining a first loop structure and second loop structure, the first loop structure defined by the first end of the first support member, the first end of the second support member and the crossover, the second loop structure defined by the second end of the first support member, the second end of the second support member and the crossover, a material capture structure extending within the first loop structure; and a plurality of first tissue anchors including an elongate portion and a curved portion, the curved portion configured to engage with a lumen wall. The elongate portion of the anchor has a length of at least 25% of a length between the first end and second end of the first support member. The plurality of first tissue anchors are formed on the second loop structure.

In any of the embodiments described herein the first loop structure has a first diameter and the second loop structure has a second diameter. In any of the embodiments described herein the second diameter is smaller than the first diameter. In any of the embodiments described herein the second diameter is larger than the first diameter.

In any of the embodiments described herein the plurality of first tissue anchors extend radially past the diameter of the first loop structure.

In any of the embodiments described herein the first support member and second support member have spiral configurations.

In any of the embodiments described herein the filter further comprises a plurality of second tissue anchors formed on the first loop structure. In any of the embodiments described herein the second tissue anchor has a length that is shorter than the length of the elongate portion of the plurality of first tissue anchors.

In any of the embodiments described herein the filters further comprise a retrieval feature projecting from the first loop structure or second loop structure.

In any of the embodiments described herein the plurality of first anchors extend radially past the first diameter and second diameter.

In any of the embodiments described herein the elongate portion has a length of at least 40% of the length between the first end and second end of the first support member. In any of the embodiments described herein the elongate portion has a length of at least 50% of the length between the first end and second end of the first support member. In any of the embodiments described herein the elongate portion has a length of at least 75% of the length between the first end and second end of the first support member.

In any of the embodiments described herein the filter has a deployed length measured in a straight line between the first end of the first support member and the second end of the first support member, the elongate portion having a length of at least 50% of the deployed length of the filter.

In some embodiments endoluminal filters are provided. The filters include a first support member having a first end and a second end, a second support member having a first end and a second end forming a crossover with the first support member, the first end of the first support member attached to the first end of the second support member, the second end of the first support member attached to the second end of the second support member, the first support member and second support member defining a first loop structure and second loop structure, the first loop structure defined by the first end of the first support member, the first end of the second support member and the crossover, the second loop structure defined by the second end of the first support member, the second end of the second support member and the crossover, a material capture structure across the first loop structure, a plurality of first tissue anchors formed on the first loop structure, and a plurality of second tissue anchors formed on the second loop structure. The first tissue anchors include an elongate portion and a curved portion configured to engage with a lumen wall. The second tissue anchors include an elongate portion and a curved portion configured to engage with the lumen wall.

In any of the embodiments disclosed herein the first loop structure has a first diameter and the second loop structure having a second diameter. In any of the embodiments disclosed herein the second diameter is smaller than the first diameter. In any of the embodiments disclosed herein the second diameter is larger than the first diameter.

In any of the embodiments disclosed herein the plurality of second tissue anchors extend radially past the diameter of the first loop structure.

In any of the embodiments disclosed herein the first support member and second support member have spiral configurations.

In any of the embodiments disclosed herein the elongate portion of the plurality of second tissue anchors has a length that is shorter than the length of the elongate portion of the plurality of first tissue anchors.

In any of the embodiments disclosed herein the elongate portion of the plurality of first tissue anchors has a length that is shorter than the length of the elongate portion of the plurality of second tissue anchors.

In any of the embodiments disclosed herein the filters further include a retrieval feature projecting from the first loop structure or second loop structure.

In any of the embodiments disclosed herein the plurality of first tissue anchors project from the first loop structure at a plurality of radial positions and the plurality of second tissue anchors project form the second loop structure at a plurality of radial positions that are different from the radial positions of the plurality of first tissue anchors.

In any of the embodiments disclosed herein the elongate portion has a length of at least 40% of a length between the first end and second end of the first support member. In any of the embodiments disclosed herein the elongate portion has a length of at least 50% of a length between the first end and second end of the first support member. In any of the embodiments disclosed herein the elongate portion has a length of at least 75% of a length between the first end and second end of the first support member.

In any of the embodiments disclosed herein the filter has a deployed length measured in a straight line between the first end of the first support member and the second end of the first support member with the elongate portion having a length of at least 50% of the deployed length of the filter.

In some embodiments methods of positioning a filter within a lumen are provided. The methods include advancing a sheath containing any of the filters disclosed herein through the lumen, deploying a portion of the filter of any of the preceding claims from the sheath into the lumen to engage the lumen wall while maintaining substantially all of the material capture of the filter within the sheath, and deploying the material capture structure of the filter from the sheath to a position across the lumen.

In any of the embodiments disclosed herein the methods further include maneuvering a snare towards the filter in the same direction used during the advancing step and engaging the snare with a filter retrieval feature positioned against a wall of the lumen.

In any of the embodiments disclosed herein the methods further include maneuvering a snare towards the filter in the opposite direction used during the advancing step, and engaging the snare with a filter retrieval feature positioned against a wall of the lumen.

In any of the embodiments disclosed herein the methods further include deploying the filter retrieval feature from the sheath after the deploying the material capture structure step.

In any of the embodiments disclosed herein the methods further include deploying a filter retrieval feature from the sheath before the deploying the material capture structure step.

In any of the embodiments disclosed herein the methods further include engaging the lumen wall with the tissue anchor attached to the filter.

In any of the embodiments disclosed herein the methods further include engaging the lumen wall with a radial force generated by the filter.

In any of the embodiments disclosed herein retrieving the filter from a femoral vein includes rotating the anchors 180 degrees.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1I illustrate various prior art filters.

FIG. 2 illustrates an endoluminal filter device in accordance with an embodiment.

FIG. 3 illustrates an endoluminal filter device in accordance with an embodiment.

FIG. 4 illustrates an endoluminal filter device in accordance with an embodiment.

FIG. 5 illustrates an endoluminal filter device in accordance with an embodiment.

FIG. 6 illustrates an endoluminal filter device in accordance with an embodiment.

FIG. 7 illustrates an endoluminal filter device in accordance with an embodiment.

FIGS. 8A-8B illustrate endoluminal filter devices in accordance with various embodiments.

FIGS. 9A-9F illustrates endoluminal filter devices in accordance with various embodiments.

FIG. 10 illustrates an endoluminal filter device in accordance with an embodiment.

FIG. 11 illustrates an endoluminal filter device in accordance with an embodiment.

FIG. 12 illustrates an endoluminal filter device in accordance with an embodiment.

FIG. 13 illustrates an endoluminal filter device in accordance with an embodiment.

FIGS. 14A-23B illustrate several alternative filtering structures.

### DETAILED DESCRIPTION

There remains a clinical need for improved endoluminal filter devices and methods. Improved endoluminal filter devices provide effective filtration over a range of lumen sizes and are easy to deploy into and retrieve from a lumen. In addition, improved endoluminal filter devices minimize thrombosis formation or tissue ingrowth on the device and are resistant to migration along the lumen. Endoluminal filters are disclosed herein with improved anchoring and filtering properties. The filters include improved anchor designs that more securely hold the filter in the desired position within the lumen. Deployment and retrieval of the anchors disclosed herein are also improved over the prior art filters. Embodiments of the filter devices of the present invention provide many and in some cases all of the features of improved endoluminal filters and have a number of uses including but not limited to: embolic protection, thrombectomy, vessel occlusion, and tethered or untethered distal protection.

The endoluminal filters can include a first support member and a second support member with opposing spiral structures and a crossover. The support members can be arranged in a configuration resembling a folded “8” or infinity symbol. A material capture structure including filter elements can span across an area of one of the loops formed by the support members. The filters can optionally include a retrieval feature configured to be captured using a snare with minimally invasive catheter techniques.

The filter includes anchor features projecting from one or both of the first support member and second support member. The anchors can include an elongate portion and a hook or barbed portion configured to engage with a lumen wall. The hook or barb portion is configured to atraumatically engage with the lumen wall during deployment, use, and retrieval. The elongate portion of the anchor can also contact the lumen wall and provide additional support to the filter during use.

The elongate anchors offer a number of advantages over prior art filter devices. The elongate anchors allow for a given filter size to treat a greater range of lumen diameters than devices with shorter anchors. The anchors can be configured to extend radially to contact a lumen wall diameter that is greater than a diameter of the filter frame and/or material capture structure. The elongate anchors can also shorten the deployed length of the filter in comparison to prior art filters. The elongate anchors can also improve retrievability. The filters with elongate anchors can be easier to grab and retrieve because less of the filter support structure contacts the lumen wall. The barbs on the elongate anchors can also withdraw from engage the lumen wall with less force than prior art anchors. In embodiments where the

In some embodiments the anchor can include breakaway barbs. The barbs can be designed to breakaway to facilitate retrieval of the filter. In some cases the breakaway barb can be made out of a biodegradable material so that the barb breaks away and then biodegrades.

In some embodiments the anchors project from the first support member and second support member forming the loop supporting the material capture structure. In some embodiments the anchors project from the loop that does not directly support the material capture structure. In some embodiments the anchors can be on each of the loops formed by the support members. In some embodiments the radial position of the anchors on the support members can be staggered between the first and second loop.

In some embodiments the elongated anchors self center the filter and the material capture structure to improve the placement and function of the filter. The self-centering feature can improve the filtering properties and make positioning, deployment, and retrieval quicker, easier, and more accurate.

In some embodiments one anchor is used. In some embodiments two or more anchors are used. In some embodiments three or more anchors are used. In some embodiments four or more anchors are used. In some embodiments five or more anchors are used. In some embodiments six or more anchors are used. In some embodiments seven or more anchors are used. In some embodiments eight or more anchors are used. In some embodiments four to eight anchors are used.

The length of the elongate portion and the length of the hook/barb portion of the anchors can vary. A ratio of the length of the generally straight elongate portion of the anchor to the length of the curved hook/barb portion of the anchor can be quantified. In some embodiments the ratio of the elongate portion to the hook portion of the anchor is greater than about 4 to 1. In some embodiments the ratio of the elongate portion to the hook portion of the anchor is greater than about 6 to 1. In some embodiments the ratio of the elongate portion to the hook portion of the anchor is greater than about 8 to 1. In some embodiments the ratio of the elongate portion to the hook portion of the anchor is greater than about 10 to 1. In some embodiments the ratio of the elongate portion to the hook portion of the anchor is greater than about 15 to 1. In some embodiments the ratio of the elongate portion to the hook portion of the anchor is greater than about 25 to 1. In some embodiments the ratio of the elongate portion to the hook portion of the anchor is greater than about 50 to 1.

The anchor length can also be quantified as a ratio between the length of the elongate portion of the anchor to a diameter of the filter loop spanned by the material capture structure. In some embodiments the ratio of the length of the elongate portion of the filter to the diameter of the filter loop is greater than about 25%. In some embodiments the ratio of the length of the elongate portion of the filter to the diameter of the filter loop is greater than about 40%. In some embodiments the ratio of the length of the elongate portion of the filter to the diameter of the filter loop is greater than about 50%.

The anchor length can also be quantified as a ratio between the length of the elongate portion of the anchor to a length of one of the support members (e.g. length of one of the spiral structures). In some embodiments the ratio of the length of the elongate portion of the filter to the length of the support member is greater than about 10%. In some embodiments the ratio of the length of the elongate portion of the filter to the length of the support member is greater than about 20%. In some embodiments the ratio of the length of the elongate portion of the filter to the length of the support member is greater than about 25%. In some embodiments the ratio of the length of the elongate portion of the filter to the length of the support member is greater than about 30%. In some embodiments the ratio of the length of the elongate portion of the filter to the length of the support member is greater than about 40%. In some embodiments the ratio of the length of the elongate portion of the filter to the length of the support member is greater than about 50%.

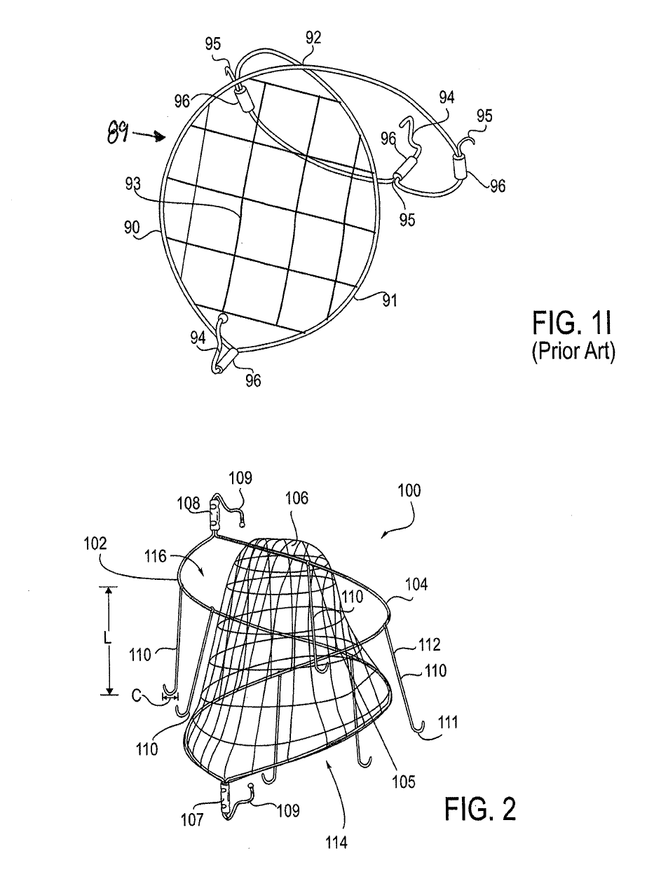

The anchor length can also be quantified as a ratio between the length of the elongate portion of the anchor to a length of the deployed filter. The length of the deployed filter can be measured from filter end to filter end. FIG. 2 shows a filter in a deployed configuration with the deployed length of the filter being the straight line distance from end 107 to end 108. In some embodiments the ratio of the length of the elongate portion of the filter to the length of the deployed filter is greater than about 50%. In some embodiments the ratio of the length of the elongate portion of the filter to the length of the deployed filter is greater than about 75%. In some embodiments the ratio of the length of the elongate portion of the filter to the length of the deployed filter is greater than about 100%.

In some embodiments the filter is configured such that both of the loops formed by the support members have a diameter that is less than the diameter of the lumen. In these embodiments the anchors can extend radially from the filter support members to contact the lumen walls and hold the filter in place. The anchors can be configured to center the filter loop supporting the material capture structure. These configurations allow for a given filter size to be deployed in a greater range of lumen diameters than prior art filters.

In some embodiments the filter loop supporting the material capture structure is designed to have a diameter that is less than the diameter of the lumen such that the material capture structure spans less than the entire diameter of the lumen. In these embodiments the filter is positioned to filter a central area of the lumen. In such embodiments the anchors can radially extend from the support members to securely hold the material capture structure and support frame in place along with centering the material capture structure. The second loop can also have a diameter that is less than the diameter of the lumen. In the alternative the second loop can have a diameter that is sized to contact the lumen walls to provide additional support to the loop holding the material capture structure.

In some embodiments the support members can be modified to include filter attachment points. For example the support members can have a hole, hook, loop, divot, or other structure to facilitate holding a filter element or any part of the material capture structure.

Several embodiments provide improved filtration devices that are durable, provide effective and nearly constant filter capacity over a range of lumen sizes and are easily delivered and removed from a lumen. Additionally, embodiments can be delivered into and retrieved from a lumen using minimally invasive surgical techniques approaching either end of the filter.

In some embodiments the support frame is made using a shape memory material. The shape memory material may have a pre-shaped form that ensures that the support elements are uniformly collapsible and, when deployed, provides a pre-defined range of controllable force against the lumen wall without use of hooks or barbs.

In some embodiments the material capture structure can include a plurality of filter elements. In some embodiments the material capture structure can extend tautly across the area between the support members. In some embodiments the material capture structure can have a basket configuration or windsock configuration.

The support members can be configured to collapse and expand with natural vessel movements while maintaining constant apposition with the vessel wall. One result is that the support members shape and size track to vessel movements. As a result, the filter density and capacity of embodiments of the present invention remain relatively independent of changes in vessel size. Moreover, the self centering aspect of the support structure ensures the filtration device provides uniform filtration across the vessel diameter. As such, embodiments provide generally constant filtration capacity of the device across the entire vessel lumen and during vessel contractions and expansions.

Uniform filter capacity is a significant improvement over some conventional devices. Conventional devices typically have a filter capacity that varies radially across a lumen. The radial variation in filter capacity usually results from the fact that conventional filtration elements have a generally wider spacing at the periphery of the lumen and closer spacing along the central lumen axis. The result is that larger emboli can escape along the lumen periphery. During vessel expansions and contractions, the radial variations in filter capacity are exacerbated in conventional devices.

Another advantage of some embodiments is that when released from a constrained state (i.e., within a delivery sheath), the device assumes a pre-determined form with the support members self centering the device in the vessel. The support members exert atraumatic radial force against the vessel wall to prevent or minimize device migration. In some embodiments, radial forces generated by the support members work in cooperation with the anchors to secure the device within the vessel. When device retrieval is initiated, the uniformly collapsible form of the support members cause the filter to pull away from the vessel wall as the device is being re-sheathed. The movement of the support members away from the vessel wall facilitates the atraumatic removal of the device from the vessel wall. Additionally, elongate member movement during retrieval also facilitates withdrawal of the anchors from the lumen wall.

Additional embodiments of the present invention may include a retrieval feature on one or both ends of the device. The use of retrieval features on both ends of the device allows deployment, repositioning and removal of the device to be accomplished from either end of the device. As a result, the use of retrieval features on both ends of the device enables both antegrade or retrograde approaches to be used with a single device. The retrieval feature may be integral to another structural member or a separate component. In some embodiments, the retrieval feature is collapsible and may have a curved shape or a generally sinusoidal shape.

The filters disclosed herein can be made out of any biocompatible material. Examples of biocompatible materials include shape memory materials, biocompatible polymers, biodegradable polymers, and biocompatible materials. In one embodiment, the support members are formed from MRI compatible materials. The support members preferably contain no sharp bends or angles to produce stress risers that may lead to fatigue issues, vessel erosion, and facilitate device collapse.

Examples of suitable shape memory alloy materials include, for example, copper-zinc-aluminium, copper-aluminum-nickel, and nickel-titanium (NiTi or Nitinol) alloys. Shape memory polymers may also be used to form components of the filter device embodiments of the present invention. In general, one component, oligo(e-caprolactone) dimethacrylate, furnishes the crystallizable “switching” segment that determines both the temporary and permanent shape of the polymer. By varying the amount of the comonomer, n-butyl acrylate, in the polymer network, the cross-link density can be adjusted. In this way, the mechanical strength and transition temperature of the polymers can be tailored over a wide range. Additional details of shape memory polymers are described in U.S. Pat. No. 6,388,043 which is incorporated herein by reference in its entirety. In addition, shape memory polymers could be designed to degrade. Biodegradable shape memory polymers are described in U.S. Pat. No. 6,160,084 which is incorporated herein by reference in its entirety.

Biodegradable polymers may also be used to than components of embodiments of the filter devices disclosed herein. For example, polylactide (PLA), a biodegradable polymer, has been used in a number of medical device applications including, for example, tissue screws, tacks, and suture anchors, as well as systems for meniscus and cartilage repair. A range of synthetic biodegradable polymers are available, including, for example, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(DTH iminocarbonate), poly(bisphenol A iminocarbonate), poly(ortho ester), polycyanoacrylate, and polyphosphazene. Additionally, a number of biodegradable polymers derived from natural sources are available such as modified polysaccharides (cellulose, chitin, dextran) or modified proteins (fibrin, casein). The most widely used compounds in commercial applications include PGA and PLA, followed by PLGA, poly(e-caprolactone), polydioxanone, trimethylene carbonate, and polyanhydride. Additional polymers that can be used include poly(amino acids) such as poly(L-glutamate), poly(L-lysine), and poly(L-leucine). Biodegradable polyurethane based materials can also be used.

In some embodiments non-polymeric materials can be used. In some embodiments non-shape memory materials are used. For example, magnesium and other biocompatible metals can also be used.

Any of the filter devices disclosed herein can be deployed and retrieved using minimally invasive catheter techniques. In some embodiments the material capture side of the filter is deployed first. In some embodiments the material capture side of the filter is deployed second. In some embodiments the deployed filter is captured using a retrieval feature on one of the ends of the filter. Contacting the retrieval feature on the filter can cause the filter to collapse. The collapsed filter can then be retrieved through the catheter. In some embodiments retrieving the filter from the femoral vein includes rotating the anchors by 180 degrees during retrieval of the filter.

Various features shown in the embodiments illustrated in the figures are now referenced. FIG. 2 illustrates an endoluminal filter device in accordance with an embodiment. The filter 100 includes a first support member 102 and second support member 104. The first support member 102 and second support member 104 are arranged to form a crossover 105 forming a first loop 114 and a second loop 116. The first support member 102 and second support member 104 are connected at ends 107, 108. A material capture structure 106 is supported by the first loop 114. The filter 100 includes retrieval features 109. The filter 100 includes anchors 110. The anchors 110 are connected to both of the support members and the areas of the support members forming the first loop 114 and second loop 116. The anchors 110 include a generally straight elongate portion 112 having a length L and a curved hook portion 111 having a length C. The material capture structure 106 includes a plurality of elements spaced to catch material flowing through the lumen. The filter 100 illustrated in FIG. 2 has a windsock type configuration.

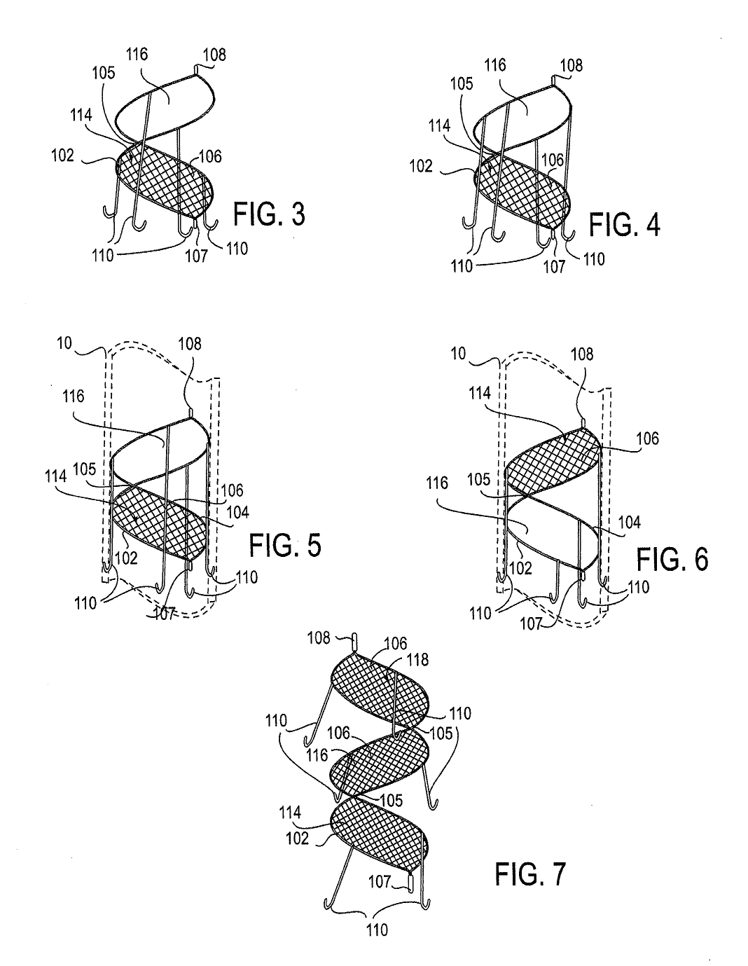

FIGS. 3 and 4 illustrate embodiments of endoluminal filter devices. FIG. 3 illustrates a filter 100 with a material capture structure 106 extending tautly across the first loop 114 of the filter. Two anchors 110 project from the first loop 114 on roughly opposing sides of the loop. Two anchors 110 project from the second loop 116 on roughly opposing sides of the loop. The position of the anchors 110 extending from the first loop 114 are radially offset from the position of the anchors 110 extending from the second loop. FIG. 4 illustrates a filter similar to FIG. 3 but with all four anchors 110 extending from the second loop 116. The four anchors 110 are roughly evenly spaced about the diameter of the second loop 116.

FIGS. 5 and 6 illustrate embodiments of endoluminal filter devices within a lumen 10. The direction of fluid flow through the lumen is illustrated by the arrow. The filter 100 illustrated in FIG. 5 has the material capture structure 106 on the upstream side of the filter 100. FIG. 5 illustrates four anchors 110 projecting from the second loop 116. In contrast to FIG. 5, FIG. 6 illustrates a filter with the material capture structure 106 on the second loop 116, which is on the downstream side of the filter 100. FIG. 5 illustrates two anchors 110 projecting from each of the first loop 114 and second loop 116 with a radial offset.

FIG. 7 illustrates an endoluminal filter device in an embodiment having three loops. The filter 100 has two crossovers 105 forming a first loop 114, second loop 116, and a third loop 118. The filter 100 is illustrated with material capture structures 106 spanning each of the three loops. In some embodiments the material capture structure spans only one of the loop structures. The filter has two anchors 110 projecting from each of the first loop 114, second loop 116, and third loop 118. The anchors 110 projecting from the first loop 114 and third loop 118 are radially offset from the anchors 110 projecting from the second loop 116.

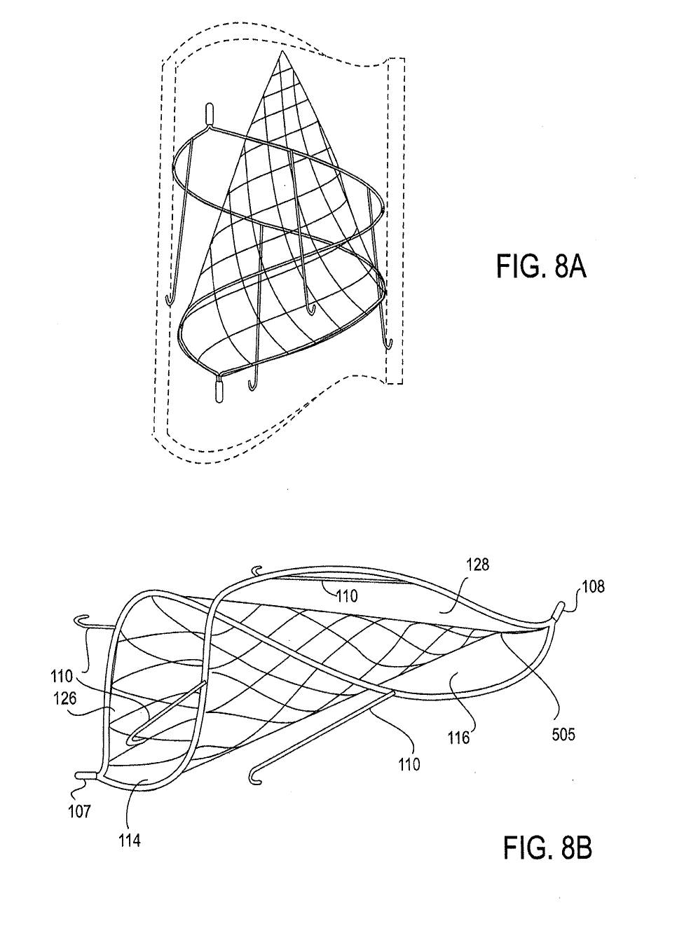

FIGS. 8A-8B illustrate endoluminal filter devices in accordance with various embodiments. The filter 100 illustrated in FIG. 8A has a material capture structure 106 with a windsock configuration and four anchors 110 projecting from the second loop 116.

FIG. 8B illustrates a material capture structure 106 with a windsock configuration and the first support member 102 and second support member 104 having a longer filter shape with the second loop 116 having an elliptical shape. Two anchors 100 project from the first loop 114 and two anchors 110 project from the second loop 116 at similar radial positions.

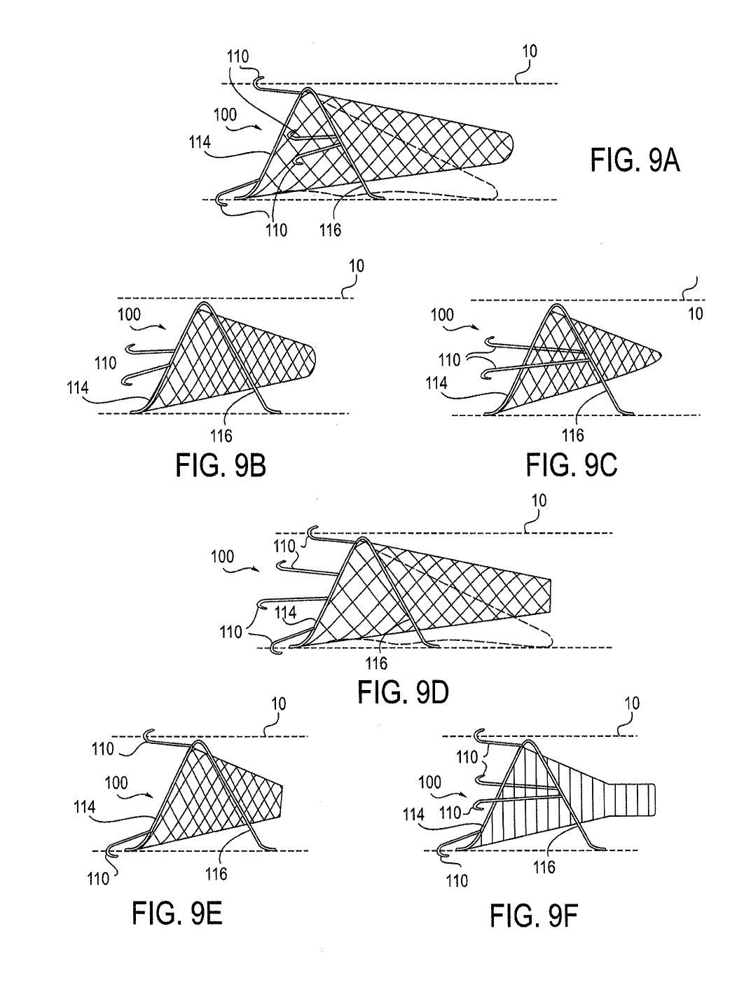

FIGS. 9A-9F illustrates endoluminal filter devices in accordance with various embodiments. The filters illustrated in FIGS. 9A-9F illustrate material capture structures having different windsock type shapes and anchor configurations. FIGS. 9A and 9F illustrate filters 100 with two anchors 110 projecting from each of the first loop 114 and second loop 116 with the anchors radially offset between the two loops. FIGS. 9B and 9E illustrate filters 100 with two anchors 110 projecting from only the first loop 114. FIG. 9C illustrates a filter 100 with two anchors 110 projecting from only the second loop 116. FIG. 9D illustrates a filter 100 with four anchors 110 projecting from only the first loop 114.

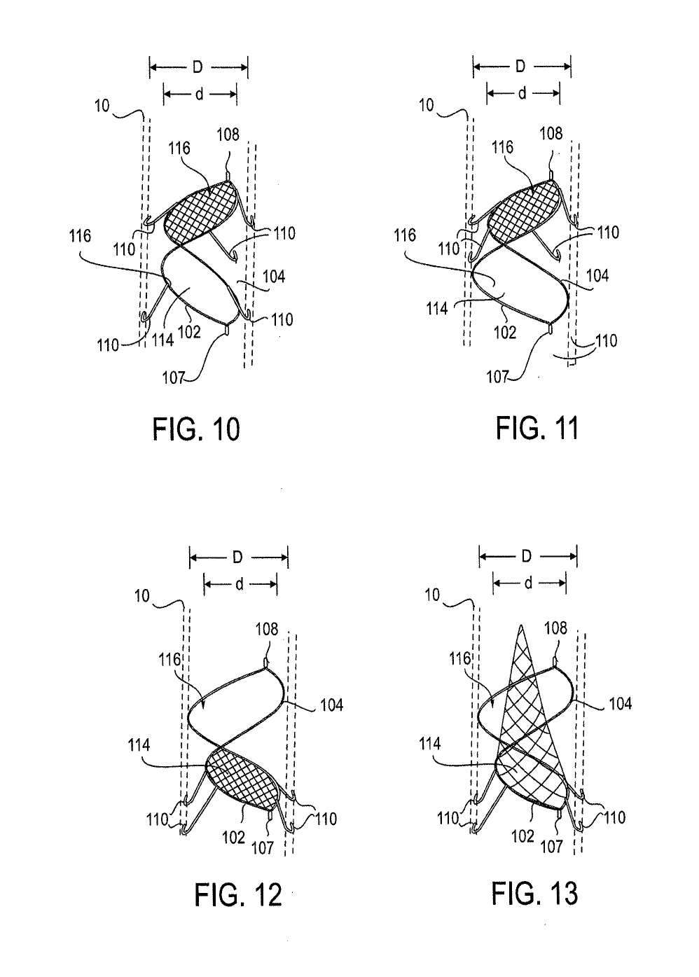

FIG. 10 illustrates an endoluminal filter device 100 within a lumen 10 having a diameter D. The first loop 114 and second loop 116 have a diameter d that is less than the diameter D of the lumen. The anchors 110 extend outward from both of the first loop 114 and second loop 116 to engage with the lumen 10 wall to hold the filter 100 centered and in place within the lumen.

FIG. 11 illustrates an endoluminal filter device 100 within a lumen 10 having a diameter D. The second loop 116 has a diameter d that is less than the diameter D of the lumen. The anchors 110 extend outward from the second loop 116 to engage with the lumen 10 wall to hold the filter 100 centered and in place within the lumen. The first loop 114 has a diameter that is larger than the diameter of the second loop 116 and is sized to engage with the lumen 10 wall to provide additional support for the filter. In FIG. 11 the material capture structure 106 spans the second loop 116 and is on the downstream side of the filter.

FIG. 12 illustrates an endoluminal filter device 100 within a lumen 10 having a diameter D. The first loop 114 has a diameter d that is less than the diameter D of the lumen. The anchors 110 extend outwardly from the first loop 114 to engage with the lumen 10 wall to hold the filter 100 centered and in place within the lumen. The second loop 116 has a diameter that is larger than the diameter of the first loop 114 and is sized to engage with the lumen 10 wall to provide additional support for the filter. In FIG. 12 the material capture structure 106 spans the first loop 116 and is on the upstream side of the filter.

FIG. 13 is similar to the filter in FIG. 12 but with longer anchors 110 and a material capture structure 106 in a windsock configuration.

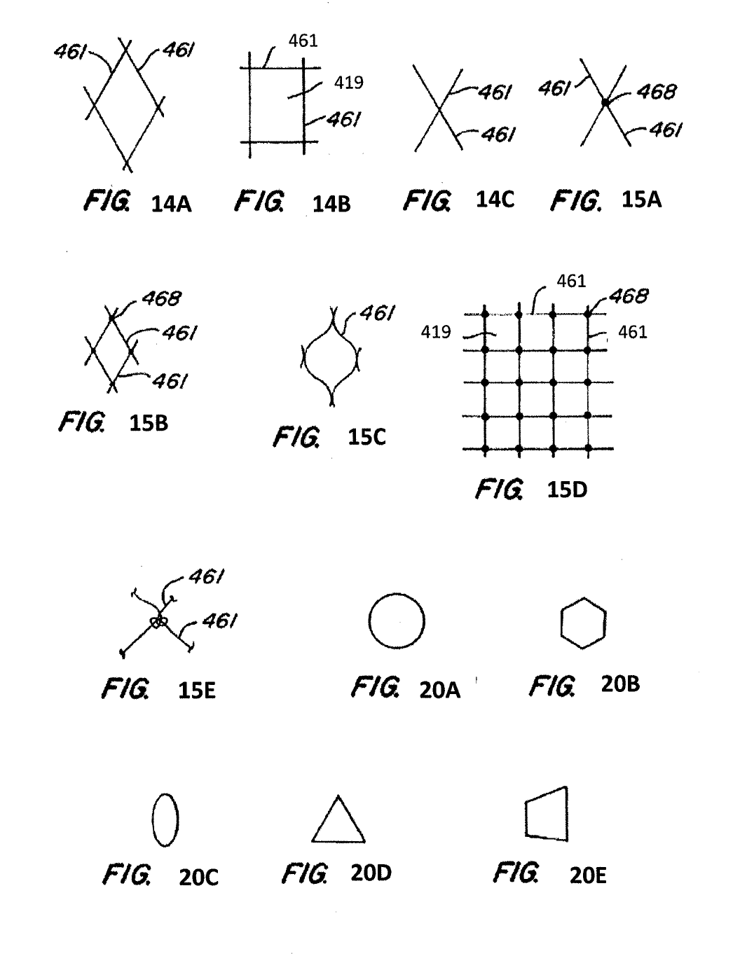

The material capture structures illustrated in FIGS. 14A-23B can be used in any of the embodiments of endoluminal filters described herein. The material capture structure can be selected based on the desired application for the filter. For example if the filter is for arterial and/or distal protection then a fine material capture structure could be used. In another example, if the filter is for embolic protection then the material capture structure can be occlusive.

In some embodiments, the material capture structure contains a number of filter cells. Filter cells may be formed in a number of different ways and have a number of different shapes and sizes. The shape, size and number of filter cells in a specific filter may be selected based on the use of a particular filter. For example, a filter device of the present invention configured for distal protection may have a filter cell size on the order of tens to hundreds of microns to less than 5 millimeters formed by a selecting a filter material with a pore size (FIGS. 15A, 15B) suited to the desired filtration level. In other applications, the filter cell may be formed by overlapping (i.e., joined or crossed without joining) filaments to form cells that will filter out debris in a lumen above a size of 2 mm. Various other filter sizes and filtration capacities are possible as described herein.

Intersecting filaments (FIG. 14C) may be used to form diamond shaped filter cells (FIG. 14A), as well as rectangular shaped filter cells 419 (FIG. 14B). Multiple strand patterns may also be used such as the three strand 461a, 461b and 461c array illustrated in FIG. 25B. Intersecting filaments may also be knotted, tied or otherwise joined 468 (FIGS. 15A and 15E). Intersecting filaments may form the same or different filter cell shapes such as, for example, an elongated oval in FIG. 15C, one or more joined diamonds as in FIG. 15B and an array of joined polygons as in FIG. 15D. In one embodiment, a filter cell is defined by at least three intersecting filaments 461. The filter element 461 may be formed from any of a wide variety of acceptable materials that are biocompatible and will filter debris. For example, filaments, lines and strands described herein may be in the form of a multifilament suture, a monofilament suture a ribbon, a polymer strand, a metallic strand or a composite strand. Additionally, filaments, lines and strands described herein may be formed from expanded polytetrafluoroethylene (ePTFE), polytetrafluoroethylene (PTFE), Poly(ethylene terephthalate) (PET), Polyvinylidene fluoride (PVDF), tetrafluoroethylene-co-hexafluoropropylene (FEP), or poly(fluoroalkoxy) (PFA), other suitable medical grade polymers, other biocompatible polymers and the like.

The joined polygons may have any of the shapes illustrated in FIGS. 20A-20F. It is to be appreciated that filter cells may have any, one or more, or hybrid combinations of shapes such as, for example, circular (FIG. 20A), polygonal (FIG. 20B), oval (FIG. 20C), triangular (FIG. 20D), trapezoidal or truncated conical (FIG. 20E).

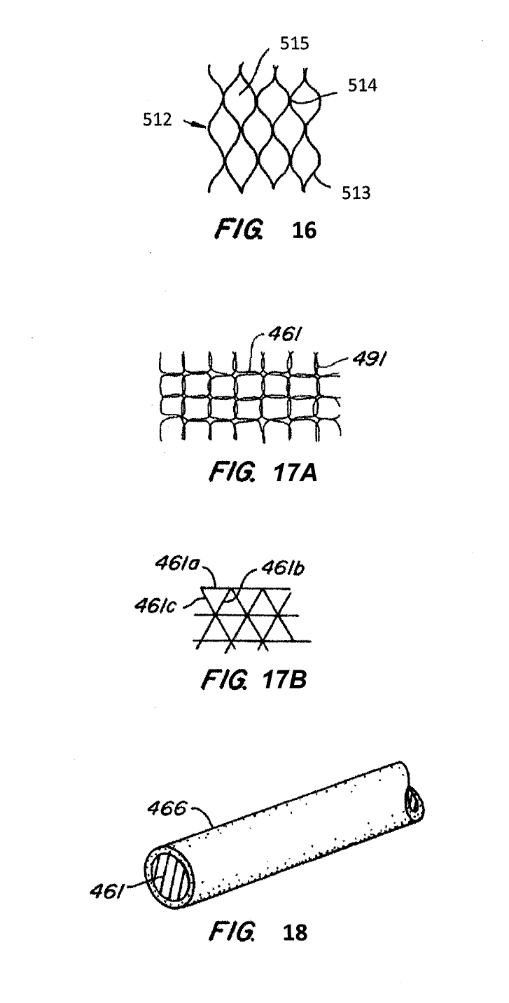

In addition, the material capture structure may have filter cells formed by extruding a material into a material capture structure. FIG. 16 illustrates an exemplary filtering structure 512 where a material is extruded into strands 513 that are joined 514 and spaced apart for form one of more filter cells 515. In one embodiment, the strands are extruded from Polypropylene material, forming diamond shaped filter cells approximately 4 mm in height and 3 mm in width.

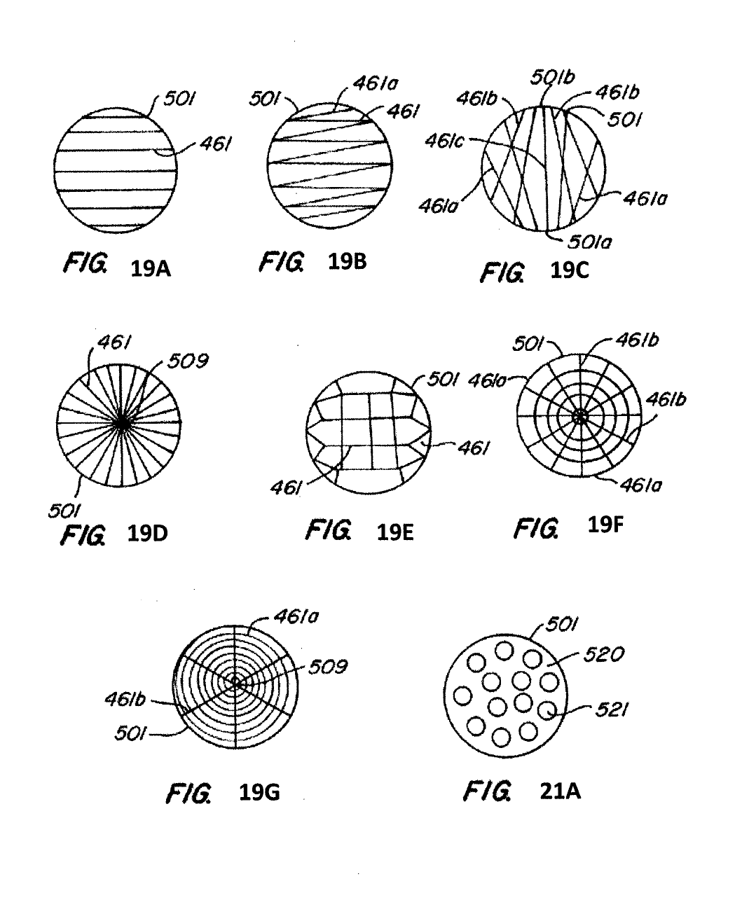

FIGS. 19A-23B illustrate several different filtering structure configurations. For simplicity of illustration, the filtering material is shown attached to a circular frame 501. It is to be appreciated that the circular frame 501 represents any of the various open loop, rounded frame or other support frames described herein. FIG. 19A illustrates a frame pattern similar to FIG. 22D. FIG. 19B adds an additional transverse filaments 461a at an angle to the filaments 461. FIG. 19C illustrates a plurality of filaments 461a extending up from the frame bottom 501a about a central filament 461c and a plurality of filaments 461b extending down from the frame top 501b about a central filament 461c. In this illustrative embodiment, the filaments 461a, b are arranged symmetrically about the central filament 461c. Other non-symmetrical configurations are possible. More than one central filament 461c may be used to form a variety of different size and shaped polygonal filter cells (e.g., FIG. 19E).

Filaments may also be arranged using a variety of radial patterns. Fr example, multiple filaments 461 may from a common point 509 out the edge of frame 501. In some embodiments, the common point is central to the frame 501 (FIG. 19D) and in other embodiments the common point 509 is in a different, non-central location. The sectors formed by the multiple filaments (FIG. 19D) may be further divided into multiple filter cell segments by winding a filament 461a about and across segment filaments 461b. In contrast to a single filament spirally out from the point 509 as in FIG. 19G, the segmented filter cells in FIG. 19F are formed by attaching single filament 461a to the segment filaments 461b.

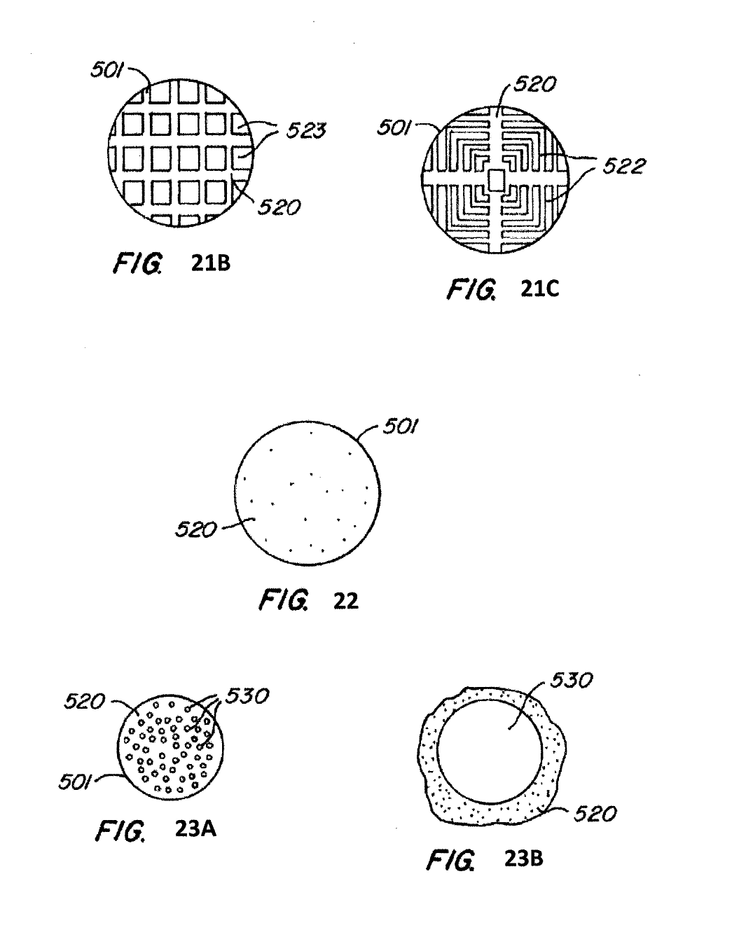

FIGS. 21A-C and FIG. 22 illustrate the use of a sheet of material 520 to form a filter structure. The material 520 may have any of a variety of shapes formed in it using any suitable process such as punching, piercing, laser cutting and the like. FIG. 21A illustrates a circular pattern 521 formed in material 520. FIG. 21B illustrates a rectangular pattern 523 formed in material 520. FIG. 30C illustrates a complex pattern 522 cut into material 522. It is to be appreciated that the material 520 may also be placed in the frame 501 without any pattern (FIG. 22). The illustrative embodiment of FIG. 22 may be useful for occluding the flow within a lumen. Suitable materials 520 for an occlusion application include for example, wool, silk polymer sheets, other material suited to prevent blood flow in a lumen when extended across a lumen and the like. Additionally, the filter material 520 may be a porous material having pores 530 (FIG. 23A). The material 520 may be selected based on the average size of individual pores 530 (FIG. 23B) depending upon the procedure or use of the filter device. For example, the material 520 may be any of the porous materials using in existing distal protection and embolic protection devices. In general, a wide variety of pore 530 sizes are available and may range from 0.010″ to 0.3″. Other pore sizes are also available depending upon the material 520 selected.

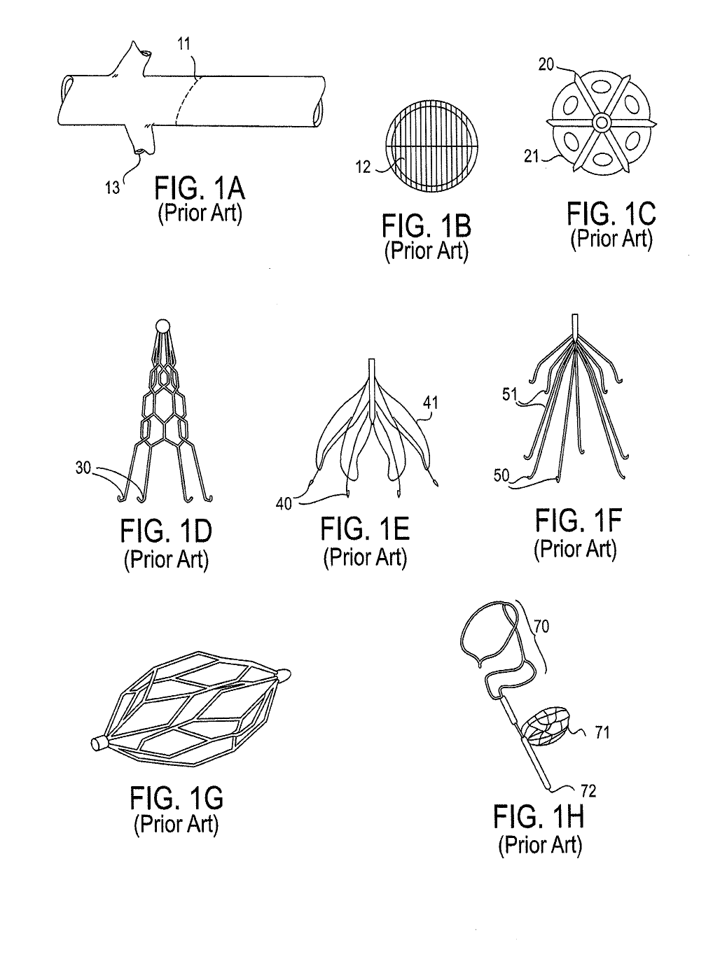

FIGS. 14-23B illustrate the use of nets or other web structures within the filtering device. The various net structure embodiments described herein are used as material capture structures within filter device embodiments of the present invention. Each of these alternative is illustrated in a support structure similar to that of device 89 in FIG. 1I and elsewhere; however, any of the alternatives can also be used with all of the embodiments of filter designs disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An endoluminal filter, comprising:
a first support member having a first end and a second end;
a second support member attached to the first end of the first support member or the second end of the first support member and forming a crossover with the first support member;
a material capture structure extending between the first and second support members, the crossover, and the first end or the second end of the first support member; and
at least one tissue anchor including an elongate portion and a curved portion, the curved portion configured to engage with a lumen wall, the elongate portion having a length of at least 25% of a length between the first end and second end of the first support member, the at least one tissue anchor formed on the first support member or the second support member.

2. An endoluminal filter according to claim 1 further comprising a plurality of second tissue anchors having a curved portion and an elongate portion with the elongate portion shorter than the elongate portion of the at least one tissue anchor.

3. An endoluminal filter according to claim 1 wherein the crossover forms a first loop between the first support member and second support member and a second loop between the first support member and second support member.

4. An endoluminal filter according to claim 3 wherein the first loop has a first diameter and the second loop has a second diameter, the second diameter smaller than the first diameter, wherein the anchors are formed on the support members forming the second loop.

5. An endoluminal filter according to claim 4 wherein the length of the elongate portion is greater than about one-quarter of the second diameter.

6. An endoluminal filter according to claim 5 wherein the length of the elongate portion is greater than about one-half of the second diameter.

7. An endoluminal filter according to claim 4 wherein the filter is configured to be placed in a lumen having a lumen diameter, wherein the lumen diameter is greater than the first diameter and second diameter.

8. An endoluminal filter according to claim 7 wherein the anchors are configured to extend radially from the first and second support member to contact the lumen.

9. An endoluminal filter according to claim 1 wherein the material capture structure has a windsock configuration

10. An endoluminal filter according to claim 1 wherein the material capture structure extends within a plane defined by the first and second support members, the crossover, and the first end or the second end of the first support member.

11. An endoluminal filter according to claim 1 further comprising a second crossover between the first support member and second support member.

12. An endoluminal filter according to claim 1 wherein the second support member is attached to the first end of the first support member and the second end of the first support member.

13. An endoluminal filter according to claim 1 wherein the first support member and the second support member are formed from a single wire.

14. An endoluminal filter according to claim 1 wherein the first support member or the second support member forms a retrieval feature.

15. An endoluminal filter according to claim 1 further comprising:
a retrieval feature on the first end and a retrieval feature on the second end.

16. An endoluminal filter according to claim 1 wherein the first support member and second support member comprise one or more divots configured to engage with the material capture structure.

17. An endoluminal filter of claim 1 wherein the elongate portion has a length of at least 40% of the length between the first end and second end of the first support member.

18. An endoluminal filter of claim 1 wherein the elongate portion has a length of at least 50% of the length between the first end and second end of the first support member.

19. An endoluminal filter of claim 1 wherein the elongate portion has a length of at least 75% of the length between the first end and second end of the first support member.

20. An endoluminal filter of claim 1 wherein the filter has a deployed length measured in a straight line between the first end of the first support member and the second end of the first support member, the elongate portion having a length of at least 50% of the deployed length of the filter.

21. An endoluminal filter, comprising:
a first support member having a first end and a second end;
a second support member having a first end and a second end forming a crossover with the first support member, the first end of the first support member attached to the first end of the second support member, the second end of the first support member attached to the second end of the second support member,
the first support member and second support member defining a first loop structure and second loop structure, the first loop structure defined by the first end of the first support member, the first end of the second support member and the crossover, the second loop structure defined by the second end of the first support member, the second end of the second support member and the crossover;
a material capture structure extending within the first loop structure; and
a plurality of first tissue anchors including an elongate portion and a curved portion, the curved portion configured to engage with a lumen wall, the elongate portion having a length of at least 25% of a length between the first end and second end of the first support member, the plurality of first tissue anchors formed on the second loop structure.

22. An endoluminal filter according to claim 21 wherein the first loop structure having a first diameter and the second loop structure having a second diameter.

23. An endoluminal filter according to claim 22 wherein the second diameter is smaller than the first diameter.

24. An endoluminal filter according to claim 22 wherein the second diameter is larger than the first diameter.

25. An endoluminal filter according to claim 23 wherein the plurality of first tissue anchors extend radially past the diameter of the first loop structure.

26. An endoluminal filter according to claim 21:
further comprising a plurality of second tissue anchors formed on the first loop structure.

27. An endoluminal filter according to claim 26 wherein the second tissue anchor has a length that is shorter than the length of the elongate portion of the plurality of first tissue anchors.

28. An endoluminal filter according to claim 21 further comprising a retrieval feature projecting from the first loop structure or second loop structure.

29. An endoluminal filter according to claim 22 wherein the plurality of first anchors extend radially past the first diameter and second diameter.

30. An endoluminal filter of claim 21 wherein the elongate portion has a length of at least 40% of the length between the first end and second end of the first support member.

31. An endoluminal filter of claim 21 wherein the elongate portion has a length of at least 50% of the length between the first end and second end of the first support member.

32. An endoluminal filter of claim 21 wherein the elongate portion has a length of at least 75% of the length between the first end and second end of the first support member.

33. An endoluminal filter of claim 21 wherein the filter has a deployed length measured in a straight line between the first end of the first support member and the second end of the first support member, the elongate portion having a length of at least 50% of the deployed length of the filter.
